(19) United States
(12) Patent Application Publication
(10) Pub. No.: US 2006/0233730 A1
(43) Pub. Date: Oct. 19, 2006

(54) Hyperbranched polymers with low glass-transition temperature and uses thereof in cosmetics

(76) Inventor: Nathalie Mougin, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/544,040

(22) PCT Filed: Dec. 12, 2003
(86) PCT No.: PCT/FR03/03697

§ 371 Date: Jan. 10, 2006

Related U.S. Application Data
(60) Provisional application No. 60/448,113, filed on Feb. 20, 2003.

(30) Foreign Application Priority Data
Jan. 31, 2003 (FR) 0301147

(51) Int. Cl. A61K 8/81; C08L 33/02

(52) U.S. Cl. 424/70.7; 424/70.16; 525/221

(57) ABSTRACT

The invention relates to a hyperbranched polymer comprising at least three polymer branches. Said branches form either the main branch or a secondary branch and each comprise at least one branch point which is at least trivalent in order to form at least two different and independent branch points which are at least trivalent, each branch point being disposed at the core of at least one chain. The invention is characterised in that it comprises at least one polymer sequence having a glass-transition temperature (Tg) of between −150 and −20° C., preferably between −150 and −30° C. and, more preferably still, between −150 and −40° C. According to the invention, a hair-fixing and/or -setting composition comprises more than 50 wt.-% of the aforementioned polymer sequence, and preferably more than 55% or, more preferably still, more than 60%, in relation to the total weight of the polymer.

The subject of the invention is hyperbranched polymers comprising at least one polymeric sequence having a low glass transition temperature (T_{g}) and the method of manufacturing these polymers. The invention also relates to cosmetic compositions containing these polymers and to their uses, in particular in the field of hairstyle fixing and/or holding products.

“Hyperbranched polymers”, that is to say polymers comprising at least three polymeric branches, forming either the main branch or a secondary branch and each having at least one at least trivalent branching point in order to form at least two at least trivalent branching points that are separate from and independent of each other, are known. Each branching point is located within the core of at least one chain.

Within the context of the present invention, the term “trivalent branching point” is understood to mean the junction point between three polymeric branches, at least two branches of which are of different chemical composition or different structure. For example, certain branches may be hydrophilic, that is to say they may comprise predominantly hydrophilic monomers, while other branches may be hydrophobic, that is to say they comprise mostly hydrophobic monomers. Other branches may also form a random polymer or a block polymer.

Hyperbranched polymers comprising at least three polymeric branches, forming either the main branch or a secondary branch and each having at least one at least trivalent branching point in order to form at least two at least trivalent branching points that are separate from and independent of each other, are preferred. Each branching point is located within the core of at least one chain.

By analogy, the term “at least trivalent branching point” is understood to mean the junction points between at least three polymeric branches, for example n polymeric branches, at least n-1 branches of which are of different chemical composition or different structure.

The term “core of a chain” is understood to mean the atoms located within the polymeric chain, excluding the atoms forming the two ends of this chain.

The definition according to the invention of the concept of a “hyperbranched polymer” does not, in particular, include:

branched polymers or graft polymers, that is to say polymers consisting of a main chain having multiple trivalent branching points, each of which is the starting point for a linear side chain, it being possible for the side chains to consist of one or more blocks, these side chains being by nature identical to or different from the main chain, such as for example those described in document EP 815 848, which relates to copolymers with a carbon/fluorine backbone of T_{g} between 0 and 30° C. and to rigid graft copolymers of T_{g} above 25° C., or in document WO 97/35541, which relates to copolymers with a rigid, hydrophilic, vinyl/acrylic backbone and to hydrophobic flexible graft copolymers;

comb polymers (a special case of graft polymers), that is to say polymers consisting of a main chain having multiple trivalent branching points, each of which is the starting point of a linear side chain (Glossary of Basic Terms in Polymer Science/IUPAC/1996), the branching points being located at regular intervals; and

star polymers, that is to say those in which all the branching points are located at a single point.

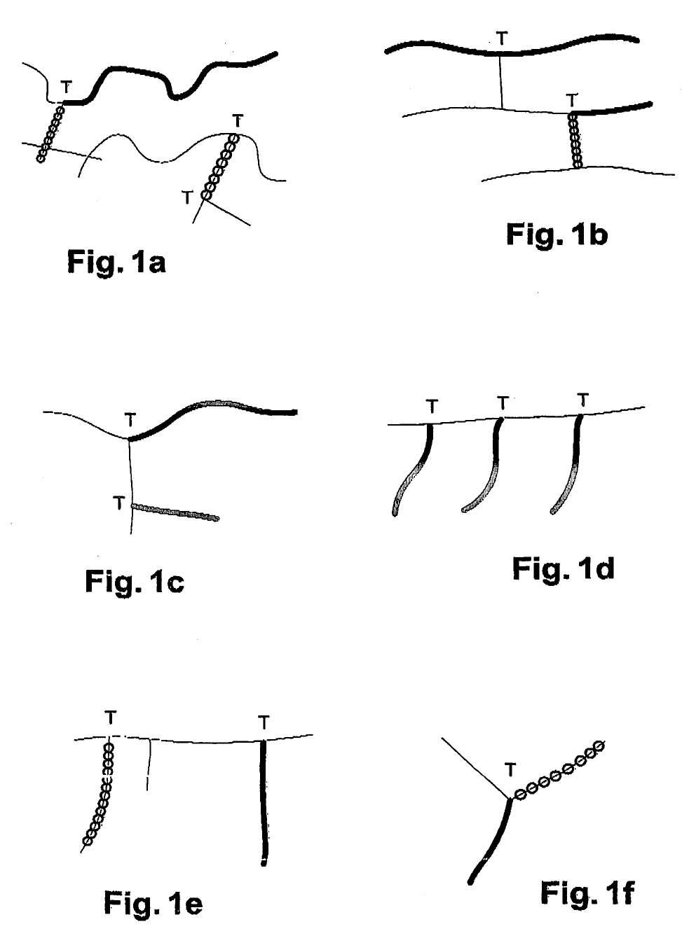

FIG. 1 appended hereto is intended to make it easier to understand the concept of a “hyperbranched polymer”. It shows:

FIG. 1a: a first example of a hyperbranched structure;

FIG. 1b: a second example of a hyperbranched structure;

FIG. 1c: a minimal hyperbranched structure;

FIG. 1d: a comb polymer structure;

FIG. 1e: a branched polymer structure; and

FIG. 1f: a star polymer structure.

In the figures, the point “T” represents the trivalent point. The fine lines, the bold lines and the lines formed by a succession of circles represent polymeric branches of different chemical composition or different structure.

According to the invention, the glass transition temperature (T_{g}) of the polymer is measured by DMTA (Dynamic and Mechanical Temperature Analysis).

To measure the glass transition temperature (T_{g}) of the polymer, viscoelasticity tests are performed using a DMTA apparatus from Polymer Laboratories, on a polymer film specimen about 150±50 μm in thickness, 5 mm in width and 10 mm in length, after drying for 24 hours at 23° C. and at 50-55% relative humidity. A tensile stress is imposed on this specimen. The specimen undergoes a static force of 0.01 N on which is superposed a sinusoidal displacement of ±8 μm at a frequency of 1 Hz. The specimen is thus deformed in the linear range, at low strain levels. This tensile stress is applied to the specimen at temperatures varying from −150° C. to +220° C., with the temperature varying at 3° C. per minute.

The complex modulus E*=E′+iE″ of the polymer tested is then measured as a function of temperature.

From these measurements, the dynamic storage modulus E′ and dynamic loss modulus E″, and also the damping factor tan δ(=E″/E′) are deduced.

The curve of the tan δ values is then plotted as a function of temperature—this curve shows at least one peak. The glass transition temperature T_{g} of the polymer corresponds to the temperature at which the top of this peak occurs.

When the curve has at least two peaks (in this case, the polymer has at least two (T_{g}s), the value of T_{g} of the polymer tested will be taken as the temperature for which the curve has the peak of highest amplitude (that is to say corresponding to the highest value of tan δ—in this case, only the “predominant” T_{g} will be taken as the value of T_{g} of the polymer tested).

Hyperbranched polymers and their method of manufacture and their use in hair cosmetics are, for example, described in international application WO 01/96429.

This international application WO 01/96429 relates to hyperbranched block polymers prepared from a first type of acrylic monomer and from branching monomers possessing two polymerizable functions of different reactivities, so as to obtain polymers that include pendent allyl units, which may be subsequently polymerized, in the presence of a second type of acrylic monomer.

However, the polymers described in this prior application provide too little an improvement in the retention and/or holding of the hairstyle.

In the cosmetics field, it is often sought to have compositions for obtaining a coating, especially an adhesive or film-forming one, on the keratinous materials in question, such as the hair, skin, eyelashes or nails.

In particular, these compositions may provide color (makeup or hair dye compositions), a glossy or matt appearance (skin makeup or care compositions), physical properties, such as shaping properties (hair, especially hairstyling, compositions) and care or protection properties.

It is generally sought to achieve good remanence and staying power of the cosmetic coating, and also good adhesion to the support. In particular, it is desirable for this coating to be able to resist mechanical attack, such as rubbing and transfer by contact with another object, and resist water, sweat, tears, rain, sebum and oils. This is particularly true in the case of makeup, especially in the field of lipsticks in which it is desirable to achieve prolonged retention of the color and gloss and for the color not to be transferred; in the field of foundations, eyeshadows and powders, in which it is desirable for the color provided to be retained, while keeping the matt character of the original tint for as long as possible despite the secretion of sebum and sweat, and also to prevent transfer.

Furthermore, the makeup compositions must be comfortable to wear and must not have too tacky a texture.

The problem posed by the invention is to find polymers that provide fibers with a natural and durable cosmetic and are capable of forming a sufficiently homogeneous and resistant film.

Surprisingly and unexpectedly, the Applicant has now discovered that certain hyperbranched polymers, which meet very specific demands in terms of glass transition temperature (T_{g}), fully meet the requirements indicated above.

One subject of the invention is hyperbranched polymers comprising at least three polymeric branches, forming either the main branch or a secondary branch and each having at least one at least trivalent branching point in order to form at least two at least trivalent branching points that are separate from and independent of each other, each branching point being located within the core of at least one chain, and comprising at least one polymeric sequence whose glass transition temperature (T_{g}) is between −150 and −20° C., preferably between −150 and −30° C. and more preferably between −150 and −40° C., the relative proportion by weight of this polymeric sequence being greater than 50%, preferably greater than 55% and even more preferably greater than 60% relative to the total weight of the polymer.

Without being tied down by theory, the Applicant believes that the greater than 50% by weight proportion of polymeric sequences having a low T_{g} is responsible for the formation of a film that can constitute a flexible layer around the hair or a flexible “mesh” on the skin.

Preferably, the polymer according to the invention furthermore includes at least one polymeric sequence whose glass transition temperature (T_{g}) is between 20 and 150° C. and preferably between 20 and 100° C.

Advantageously, the polymer according to the invention includes crosslinking points forming between 0.1 and 10% and preferably between 0.5 and 5% of the total number of bonds of the polymer.

The physical parameters characterizing the elastic properties of the hyperbranched polymers are especially their tensile recovery and their elongation at break:
the recovery (R given in %) is determined by a tensile creep test consisting in rapidly stretching a test specimen for a predetermined degree of elongation and then in releasing the stress and measuring the length of the test specimen. If the measurement is carried out just after the stretching, what is determined is the instantaneous recovery Rᵢ, while if it is carried out after a time t, what is determined is the recovery Rₜ;
during the tensile test, the strain at break (ε_{break}, given in %) corresponds to the maximum strain of the copolymer specimen before it breaks.

The creep test used for characterizing the hyperbranched polymers with an elastic character of the present invention is carried out as indicated below:

Firstly, it should be pointed out that these parameters relate to a film obtained by drying a solution of the copolymer in the appropriate solvent for said copolymer, for example water, at room temperature at a relative humidity close to 50%.

The mechanical properties of the film in monotonic tension are measured according to the ASTM Standards, Volume 06.01 D 2370-92 “Standard Test Method for Tensile Properties of Organic Coatings”. A speed of displacement is therefore imposed and the length (L) of the test specimen and the force (f) needed to impose this length are measured simultaneously.

As test specimen, a film of the copolymer having a thickness of 150±50 μm obtained after 48 hours of drying at 23±2° C. and 55±5% relative humidity is used. This film is obtained from a solution or dispersion containing 6% by weight of said copolymer in water or ethanol, said solution or dispersion being deposited on a Teflon (PTFE) matrix.

The test specimen is of dumbbell shape, with a gage length of 33 mm and a gage width of 6 mm.

The tests are carried out on a tensile testing machine equipped with an optical extensometer for measuring the displacement, said machine being sold under the name Zwick Z010.

The measurements are carried out under the same temperature and humidity conditions as for the drying, that is to say at a temperature of 23±2° C. and a relative humidity of 55±5%.

Each test specimen is clamped between two jaws, separated by a distance of 50±1 mm from each other, and is stretched at a rate of 50 mm/min (under the above temperature and relative humidity conditions). A speed of displacement is therefore imposed and the length (L) of the test specimen and the force (f) needed to impose this length are measured simultaneously.

The length (L) is measured with an optical extensometer using adhesive labels placed on the dumbbell test specimen. The initial distance between these two labels defines the gage length L₀ used to calculate the strain ε=(L/L₀)×100 expressed as a %.

The elastic recovery is determined as follows:

The elastic recovery (R, given in %) is determined by stretching the test specimen to an elongation of 100% (Lₘₐₓ), that is to say to twice its initial length. The stress is then released, imposing a return speed equal to the tensile stretching speed, i.e. 50 mm/minute.

The instantaneous elastic recovery (Rᵢ=εᵢ) is determined by measuring the elongation of the test specimen (expressed as a % relative to the initial length) immediately after return to a zero load. It is therefore defined by the instantaneous residual strain at zero stress: εᵢᵣ.

The instantaneous elastic recovery (Rᵢ=εᵢ) is calculated from the following formula:
Rᵢ=εᵢ=100−ε_{i, residual}

The value of the instantaneous recovery depends on many factors such as the nature, the number, the arrangement and the relative proportion of the hard and soft sequences, or the molar mass, of the polymer.

The hyperbranched polymers with an elastic character of the present invention generally have an instantaneous recovery (Rᵢ), measured under the conditions indicated above, of between 50 and 100%, preferably between 55 and 100%, better still between 55 and 95% and ideally between 60 and 95%.

To determine the 300-second recovery, the test specimen having undergone the previous operations is maintained at zero stress for a further 300 seconds and its degree of elongation as a percentage (ε₃₀₀=R₃₀₀) is measured. The 300-second recovery (R₃₀₀) in % is given by the formula below:
ε₃₀₀=R₃₀₀=100−ε_{300,residual}

The hyperbranched polymers with an elastic character of the present invention generally have a 300-second recovery (R₃₀₀), measured under the conditions indicated above, of between 55 and 100%, preferably between 60 and 100% and better still between 80 and 100%.

The copolymers of the invention are also defined by a strain or elongation at break ε_{break}, which is given in % and corresponds to the maximum strain that the copolymer specimen can undergo before it breaks.

In this case, the test is carried out until the test specimen breaks.

In addition, the hyperbranched polymer advantageously has an elongation at break of greater than 1000% and more preferably of greater than 1500%.

According to the invention, the polymer advantageously has a water solubility of greater than 1% by weight at 20° C.

A homopolymer is said to be water-soluble if it forms a clear solution when it is dissolved in an amount of 1% by weight in water at 25° C.

A homopolymer is said to be water-dispersible if, at 1% by weight in water at 25° C., it forms a stable suspension of fine, generally spherical, particles. The mean size of the particles constituting said dispersion is less than 1 μm and more generally it varies between 5 and 400 nm, preferably from 10 to 250 nm. These particle sizes are measured by light scattering.

Advantageously, the hyperbranched polymer according to the invention comprises units derived from one or more ethylenic monomers capable in particular of polymerizing by radical polymerization.

Even more advantageously, the units derived from one or more ethylenic monomers are chosen from carboxylic or sulfonic acids, such as acrylic or methacrylic acid, C₁₋₂₀ alkyl (meth)acrylates with a linear, branched, cyclic or heterocyclic chain, C₁₋₄ hydroxyalkyl (meth)acrylates, certain vinyl esters, certain vinyl ethers, styrene, certain substituted styrenes, heterocyclic monomers, optionally etherified hydroxyl-terminated monoethylene glycol, diethylene glycol or polyethylene glycol (meth)acrylates, (meth)acrylamide, certain aliphatic, cycloaliphatic or aromatic methacrylamides, (meth)acrylate or vinyl monomers with a fluorinated or perfluorinated group or (meth)acrylamides with a fluorinated or perfluorinated group, silicone-containing vinyl or (meth)acrylate monomers or silicone-containing (meth)acrylamides, acrylic or vinyl monomers having an optionally neutralized or quaternized amine function, and ethylenic carboxybetaine or sulfobetaine derivatives.

Preferably, the number-average molecular weight of the copolymer is between 5000 g/mol and 1 500 000 g/mol, especially between 5500 g/mol and 1 000 000 g/mol and better still between 6000 g/mol and 900 000 g/mol.

More preferably, the polymer according to the invention comprises hydrophilic units, the proportion by weight of which is between 5 and 70%, preferably between 5 and 65% and more preferably between 5 and 50%, these hydrophilic units being, in particular, derived from one or more ethylenic monomers.

The weight-average molecular weight (M_{w}) and number-average molecular weight (Mₙ) are determined by GPC (Gel Permeation Chromatography): THF as eluant; calibration curve obtained with linear polystyrene standards; refractometer detector.

The hyperbranched polymers according to the invention may be obtained using polymerization methods known per se by those skilled in the art.

These methods may especially comprise at least one step consisting of a radical polymerization, for example with multifunctional agents. In this case, the following may be made to react:

ethylenic monomers that can be polymerized by radical polymerization, having unsaturations, such as monomers of the (meth)acrylate, (meth)acrylamide, vinyl or allyl type; and

multifunctional compounds, that is to say those having multiple unsaturations, in proportions ranging from about 0.05 to 15%, better still from 0.05 to 12% and preferably from 0.05 to 10% by weight relative to the total weight of the compound. Preferably, the functions of the multifunctional compound have different reactivities. For example, the multifunctional compound may comprise an acrylate function and an allyl function.

The hyperbranched polymers may also be synthesized as indicated below, in which the following may be made to react:

ethylenic monomers that can be polymerized by radical polymerization, including (meth)acrylate, (meth)acrylamide, vinyl or allyl groups, indicated schematically by =. . . R;

compounds indicated schematically by =. . . A which have, apart from the unsaturation, a site capable of initiating the polymerization, after thermal activation or UV radiation or any other mechanism different from the mechanism of polymerization via the unsaturation,

which are therefore compounds having both a site capable of promoting chain propagation, that is to say having a double bond (=) and a site A capable of chain initiation.

This method is called “self-condensing vinyl polymerization”. It is described in the work “Chimie et physico chimie des polymères [Chemistry and Physical Chemistry of Polymers] by M. Fontanille and Y. Ganou, DUNOD, 2002, page 368.

To form hyperbranched polymers, the unsaturations and/or the site A capable of chain initiation may be capable of reacting according to a controlled radical polymerization mechanism, in particular an ATRP (atom transfer radical polymerization) process described for example in: Macromolecules 35, 1146-48, 2002 by J. Y. Jho and S. H. Yoo; Polym. Prepr. 37(2), 413-14, 1996; Matyjaszewski, Progress in Polymer Science, 26(3), 337-377, 2001, Annex 2.2; Chem. Review 101(12), 3737, 2001; Mueller, Macromolecules 34(18), 62026-13, 2001.

In general, the atom transfer radical polymerization is carried out by polymerizing one or more radial-polymerizable monomers in the presence of:

an initiator having at least one transferable halogen atom;

a compound comprising a transition metal capable of participating in a reduction step with the initiator and a “dormant” polymeric chain; and

a ligand that may be chosen from compounds comprising a nitrogen atom (N), an oxygen atom (O), a phosphorus atom (P) or a sulfur atom (S) that are capable of coordinating via a bond with said compound comprising a transition metal.

This process is described in particular in Application WO 97/18247 and in the article by Matyjaszewski et al., published in JACS, 117, 5614(1995).

The halogen atom is preferably a chlorine atom or a bromine atom.

In this case, the site A will, for example, be R₁—Cl, where R₁=benzene, OCOCH(CH₃)—.

Without being exhaustive, among other processes that can be used is the nitroxide method described, for example, in Chem. Review 101(12), 3681, 2001.

The nitroxide-mediated radical polymerization technique consists in blocking the radical species growing in the form of a bond of the C—O NR₁R₂ type, it being possible for R₁ and R₂ to be, independently of each other, an alkyl radical having from 2 to 30 carbon atoms or both forming, with the nitrogen atom, a ring having from 4 to 20 carbon atoms, such as for example a 2,2,6,6-tetramethylpiperidinyl ring.

This polymerization technique is described, for example, in the articles “Synthesis of nitroxy-functionalized polybutadiene by anionic polymerization using a nitroxy-functionalized terminator”, published in Macromolecules, Volume 30, pages 4238-4242, 1997, and “Macromolecular engineering via living free radical polymerizations”, published in Macromol. Chem. Phys., Vol. 199, pages 923-935, 1998, or else in Application WO-A-99/03894. In this case, the site A will, for example, be:

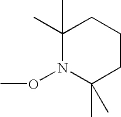

It is also possible to use:

the anionic polymerization method described, for example, in Chem. Review 101(12), 3787, 2001; and

the cationic polymerization method described for example by B. Charleux and R. Faust in Advances in Polymer Science 142, pages 1-69, 1999.

Preferably, the process takes place in two steps so as to generate sequences of different types.

Advantageously, the radical polymerization is carried out using ethylenic monomers.

The subject of the invention is also a cosmetic composition, characterized in that it comprises, in a cosmetically acceptable medium, at least one hyperbranched polymer comprising at least three polymeric branches, forming either the main branch or a secondary branch and each having at least one at least trivalent branching point in order to form at least two at least trivalent branching points that are separate from and independent of each other, each branching point being located within the core of at least one chain, and this polymer comprises at least one polymeric sequence whose glass transition temperature (T_{g}) is between −150 and −20° C., preferably between −150 and −30° C. and more preferably between −150 and −40° C., the relative proportion by weight of this polymeric sequence being greater than 50%, preferably greater than 55% and even more preferably greater than 60% relative to the total weight of the polymer.

The compositions according to the invention may form a film-forming coating, especially on hair.

Advantageously, in the compositions according to the invention, the hyperbranched polymer furthermore includes at least one polymeric sequence whose glass transition temperature (T_{g}) is between 20 and 150° C. More advantageously, the hyperbranched polymer has an instantaneous recovery of between 60 and 100% and more advantageously still the hyperbranched polymer has a 300-second recovery of between 80 and 100%.

Preferably, in the compositions according to the invention, the hyperbranched polymer has an elongation at break of greater than 1500%.

More preferably, the hyperbranched polymer has a water solubility of greater than 5% by weight at 20° C.

More preferably, the hyperbranched polymer includes a hydrophilic unit, the percentage by weight of which relative to the total weight of the polymer is between 5 and 70%, preferably between 5 and 55% and even more preferably between 5 and 50%, these being derived from one or more ethylenic monomers.

Even more preferably, the hyperbranched polymer comprises units derived from one or more ethylenic monomers.

More advantageously still, among the units derived from one or more ethylenic monomers, the following are preferred:

ethylene, isoprene and butadiene;

n-butyl acrylate, ethylhexyl acrylate, isobutyl acrylate and n-hexyl acrylate;

methoxyethyl (meth)acrylate, ethoxyethyl (meth)acrylate, POE (polyoxyethylene with the oxyethylene unit repeated 5 to 30 times) (meth)acrylate and POE (meth)acrylate-(C₁ to C₃₀)alkyl (POE) with repetition of the oxyethylene unit 5 to 30 times;

methyl vinyl ether and ethyl vinyl ether;

vinyl propionate, vinyl butyrate, vinyl ethylhexanoate, vinyl neononanoate and vinyl neododecanoate;

vinylcyclohexane, styrene and vinyl acetate;

t-butylcyclohexyl, tert-butyl, t-butylbenzyl, furfuryl and isobornyl acrylates;

methyl, ethyl, n-butyl and isobutyl methacrylates, n-hexyl, t-butylcyclohexyl, t-butylbenzyl, methoxypropyl and isobornyl methacrylates; and

N-butylacrylamide, N-t-butylacrylamide, N-isopropylacrylamide, N,N-dimethylacrylamide and N,N-dibutylacrylamide.

Among hydrophilic monomers chosen more particularly, mention may be made of:

vinylpyrrolidone, vinylcaprolactam, (meth)acrylic acid, styrenesulfonic acid, acrylamidomethylpropanesulfonic acid, vinylbenzoic acid, vinylphosphonic acid and their salts: the neutralizing agent may be a mineral base, such as LiOH, NaOH, KOH, Ca(OH)₂, NH₄OH, or an organic base, for example a primary, secondary or tertiary amine, especially an optionally hydroxylated alkylamine, such as dibutylamine, triethylamine, stearamine or else 2-amino-2-methyl-propanol, monoethanolamine, diethanolamine and stearamidopropyldimethylamine; and

C₁ to C₆ aminoalkyl (meth)acrylates and (meth)acrylamides, C₁ to C₄ N,N-dialkyl (meth)acrylates or (meth)acrylamides, such as N,N-dimethylaminoethylmethacrylate (DMAEMA), dimethylaminopropylmethacrylamide (DMAPMA) and their salts or quaternized forms. Among mineral acids, mention may be made of sulfuric and hydrochloric acid. Among organic acids, mention may be made of acids containing one or more carboxylic, sulfonic or phosphonic groups: acetic or propionic, lactic and tartric acids. The quaternizing agents may be alkyl halides such as methyl bromide or alkyl sulfates such as methyl sulfate or propane sultone.

In the compositions according to the invention, the cosmetically acceptable medium advantageously comprises one or more suitable solvents chosen from water, ketones, alcohols, alkylene glycols, alkylene glycol ethers, C₂₋₇ alkyl acetates, ethers, alkanes, aromatic hydrocarbons, aldehydes and volatile oils.

The compositions may furthermore contain a propellant gas, well known to those skilled in the art in the field of aerosols.

In the case of certain cosmetic compositions, said cosmetically acceptable medium advantageously furthermore includes a fatty phase composed of fatty substances that are liquid or solid at room temperature, of animal, plant, mineral or synthetic origin.

Advantageously, it is possible for said cosmetically acceptable medium to include one or more thickening agents, one or more anionic, cationic or nonionic, synthetic or naturally derived, film-forming polymers and/or one or more plasticizers.

Also in certain cases, said cosmetically acceptable medium furthermore includes a particulate phase consisting of pigments and/or nacres and/or fillers.

Moreover, said cosmetically acceptable medium may advantageously furthermore include one or more additives such as antioxidants, perfumes, essential oils, preserving agents, lipophilic or hydrophilic cosmetic active substances, hydrating agents, vitamins, dyes, essential fatty acids, sphingolipids, self-tanning agents, sunscreens, antifoam agents, sequestrants, antioxidants or free-radical scavengers.

The cosmetic compositions according to the invention advantageously are in the form of an organic, aqueous or hydroalcoholic lotion, suspension, dispersion or solution, optionally thickened or gelled, a mousse, a spray, an aerosol, an oil-in-water, water-in-oil or multiple emulsion, a free, compact or cast powder, a solid or an anhydrous paste.

Advantageously, the cosmetic compositions according to the invention form a hair lacquer or a hairstyling product.

Another subject of the invention is a cosmetic method, comprising the application of a composition comprising, in a cosmetically acceptable medium, at least one hyperbranched polymer comprising at least three polymeric branches, forming either the main branch or a secondary branch and each having at least one at least trivalent branching point in order to form at least two at least trivalent branching points that are separate from and independent of each other, each branching point being located within the core of at least one chain, and comprising at least one polymeric sequence whose glass transition temperature (T_{g}) is between −150 and −20° C., preferably between −150 and −30° C. and more preferably between −150 and −40° C., the relative proportion by weight of this polymeric sequence being greater than 50%, preferably greater than 55% and even more preferably greater than 60% relative to the total weight of the polymer.

Most particularly, hyperbranched polymers having a trivalent branching point, and also the cosmetic compositions comprising such hyperbranched polymers, the methods employing such polymers and their use in cosmetics, especially for the hair, are preferred.

One particularly preferred subject of the invention is the use of a hyperbranched polymer comprising at least three polymeric branches, forming either the main branch or a secondary branch and each having at least one at least trivalent branching point in order to form at least two at least trivalent branching points that are separate from and independent of each other, these branching points being located within the core of each chain, at least three polymeric branches, forming either the main branch or a secondary branch and each having at least one at least trivalent branching point in order to form at least two at least trivalent branching points that are separate from and independent of each other, each branching point being located within the core of at least one chain, comprising at least one polymeric sequence whose glass transition temperature (T_{g}) is between −150 and −20° C., preferably between −150 and −30° C. and more preferably between −150 and −40° C., the relative proportion by weight of this polymeric sequence being greater than 50%, preferably greater than 55% and even more preferably greater than 60% relative to the total weight of the polymer, in a composition intended for the setting and/or holding of a hairstyle.

The invention will be more clearly understood with the aid of the following nonlimiting example which constitutes a preferred embodiment of the polymers according to the invention.

### EXAMPLE 1

The polymer was synthesized by radical polymerization in the presence of a radical initiator of the peroxide type (tert-butylperoxypivalate), of allyl methacrylate of formula CH₂=C(CH₃)CO₂CH₂CH=CH₂, of ethylenic monomers.

The solvent was introduced into a reactor fitted with a stirrer and with a condensing system. The reactor was heated to reflux of the solvent (80° C.).

The monomers were added in two steps, so as preferably to make the first mixture (mixture 1) react before the addition of the second mixture (mixture 2). At the same time, the initiator (solution 3) was added. The temperature was maintained at reflux (80° C.), and after the end of adding mixture 2 the polymerization was continued.

The various mixtures and the solution were added using a system for controlled introduction over time.

### EXAMPLE 1 (ACCORDING TO THE INVENTION)

| | Weight | % Isopropanol/total | % Solvent/solvent + |
|---|---|---|---|
| Solvent | in g | weight of solvent | monomers |
| Isopropanol | 80 g | 80% | 50% |
| Water | 20 g | | |

| | | | | Theoretical |
|---|---|---|---|---|
| | | Weight | % in | T_{g} of |
| | Monomers | in g | mixture 1 | sequence 1 |
| Mixture 1 | Butyl | 65 | 79.8 | −30° C. |
| | acrylate | | | |
| | Acrylic acid | 15 | 18.5 | |
| | Allyl | 1.4 | 1.7 | |
| | methacrylate | | | |

| | |
|---|---|
| | %/Total |
| Weight | weight of |
| in g | monomers |

| | | | | |
|---|---|---|---|---|
| Solution 3 | Initiator: | 0.7 | g | 0.7 |
| | t-butylperoxypivalate | | | |
| | Solvent: i-PrOH/water: | 20 | g | |
| | 80/20 | | | |

| | | | % in | Theoretical |
|---|---|---|---|---|
| | | Weight | mixture | T_{g} of |
| | Monomers | in g | 2 | sequence 2 |
| Mixture 2 | Acrylic acid | 20 g | 99.25% | +100° C. |
| | Allyl | 0.15 | | |
| | methacrylate | | | |

To Summarize:

| Monomers | % in polymer |
|---|---|
| Butyl acrylate | 65 |
| Acrylic acid | 35 |
| Allyl methacrylate | 1.5 |

Mixture 1 was added over 1 h to 100 g of the solvent mixture (80/20 isopropanol/water) and then solution 3 was introduced over 4 h.

Mixture 2 was then added over a period of 2 h.

The temperature was maintained at reflux (80° C.) and the reaction was continued for 2 h.

The polymer was then precipitated in petroleum ether containing 200 g of polymer solution per 500 g of precipitant.

The polymer was dried in an oven at 40° C.

Yield 99%.

The polymer was then neutralized with 2-amino-2-methyl-propanol (AMP) in a proportion of 1 mol per mole of acid. The polymer was then dissolved in ethanol to form a 6% solution.

### EXAMPLE 2: (PRIOR ART—EXAMPLE 3 OF THE NOVEON PATENT WO 01/96429)

The same process as in Example 1 was used, except with the following percentages:

| | | Weight | % in | T_{g} of |
|---|---|---|---|---|
| | Monomers | in g | mixture 1 | sequence 1 |
| Mixture 1 | Butyl | 180 | 89.8 | −17° C. |
| | acrylate | | | |
| | Methacrylic | 17.5 | 8.7 | |
| | acid | | | |
| | Allyl | 3 | 1.5 | |
| | methacrylate | | | |

| | | | %/Total |
|---|---|---|---|
| | | Weight | weight of |
| | | in g | monomers |
| Solution 3 | Initiator: t- | 2.52 g | 0.7 |
| | butylperoxypivalate | | |
| | Solvent: i-PrOH/water: | | 0.7 |
| | 80/20 | | |

| | | | % in | Theoretical |
|---|---|---|---|---|
| | | Weight | mixture | T_{g} of |
| | Monomers | in g | 2 | sequence 2 |
| Mixture 2 | Methacrylic | 158 | 99 | +145° C. |
| | acid | | | |
| | Allyl | 1.5 | 1 | |
| | methacrylate | | | |

360 g

| Monomers | % in polymer |
|---|---|
| Butyl acrylate | 50 |
| Methacrylic acid | 48.75 |
| Allyl methacrylate | 1.25 |

1. A hyperbranched polymer comprising at least three polymeric branches, forming either the main branch or a secondary branch and each having at least one at least trivalent branching point in order to form at least two at least trivalent branching points that are separate from and independent of each other, each branching point being located within the core of at least one chain, characterized in that it comprises at least one polymeric sequence whose glass transition temperature (T_{g}) is between −150 and −20° C., preferably between −150 and −30° C. and more preferably between −150 and −40° C., the relative proportion by weight of this polymeric sequence being greater than 50%, preferably greater than 55% and even more preferably greater than 60% relative to the total weight of the polymer.

2. The polymer as claimed in claim 1, characterized in that it furthermore includes at least one polymeric sequence whose glass transition temperature (T_{g}) is between 20 and 150° C. and preferably between 20 and 100° C.

3. The polymer as claimed in either of claims 1 and 2, characterized in that its instantaneous recovery
(Rᵢ), measured under the conditions indicated above, is between 50 and 100%, preferably between 55 and 100%, better still between 55 and 95% and ideally between 60 and 95%.

4. The polymer as claimed in any one of the preceding claims, characterized in that its 300-second recovery (R₃₀₀) is between 55 and 100%, preferably between 60 and 100% and better still between 80 and 100%.

5. The polymer as claimed in any one of the preceding claims, characterized in that it has an elongation at break of greater than 1000% and preferably greater than 1500%.

6. The polymer as claimed in any one of the preceding claims, characterized in that it comprises crosslinking points forming between 0.1 and 10% and preferably between 0.5 and 5% of the total number of bonds of the polymer.

7. The polymer as claimed in any one of the preceding claims, characterized in that it has a water solubility of greater than 1% by weight at 20° C.

8. The polymer as claimed in any one of the preceding claims, characterized in that it comprises units derived from one or more ethylenic monomers.

9. The polymer as claimed in any one of the preceding claims, characterized in that its number-average molecular weight of the copolymer is between 5000 g/mol and 1 500 000 g/mol, especially between 5500 g/mol and 1 000 000 g/mol and better still between 6000 g/mol and 900 000 g/mol.

10. The polymer as claimed in any one of the preceding claims, characterized in that it comprises hydrophilic units, the proportion by weight of which is between 5 and 70%, preferably between 5 and 65% and more preferably between 5 and 50%, these hydrophilic units being, in particular, derived from one or more ethylenic monomers.

11. The polymer as claimed in claim 8 or 10, characterized in that the units derived from one or more ethylenic monomers are chosen from carboxylic or sulfonic acids, such as acrylic or methacrylic acid, C₁₋₂₀ alkyl (meth)acrylates with a linear, branched, cyclic or heterocyclic chain, C₁₋₄ hydroxyalkyl (meth)acrylates, certain vinyl esters, certain vinyl ethers, styrene, certain substituted styrenes, heterocyclic monomers, optionally etherified hydroxyl-terminated monoethylene glycol, diethylene glycol or polyethylene glycol (meth)acrylates, (meth)acrylamide, certain aliphatic, cycloaliphatic or aromatic methacrylamides, (meth)acrylate or vinyl monomers with a fluorinated or perfluorinated group or (meth)acrylamides with a fluorinated or perfluorinated group, silicone-containing vinyl or (meth)acrylate monomers or silicone-containing (meth)acrylamides, acrylic or vinyl monomers having an optionally neutralized or quaternized amine function, and ethylenic carboxybetaine or sulfobetaine derivatives.

12. The polymer as claimed in any one of the preceding claims, characterized in that the branching point is a trivalent branching point.

13. A cosmetic method, comprising the application of a composition comprising, in a cosmetically acceptable medium, at least one hyperbranched polymer comprising at least three polymeric branches, forming either the main branch or a secondary branch and each having at least one at least trivalent branching point in order to form at least two at least trivalent branching points that are separate from and independent of each other, each branching point being located within the core of at least one chain, and comprising at least one polymeric sequence whose glass transition temperature (T_{g}) is between −150 and −20° C., preferably between −150 and −30° C. and more preferably between −150 and −40° C., the relative proportion by weight of this polymeric sequence being greater than 50%, preferably greater than 55% and even more preferably greater than 60% relative to the total weight of the polymer.

14. A cosmetic composition, characterized in that it comprises, in a cosmetically acceptable medium, at least one hyperbranched polymer comprising at least three polymeric branches, forming either the main branch or a secondary branch and each having at least one at least trivalent branching point in order to form at least two at least trivalent branching points that are separate from and independent of each other, each branching point being located within the core of at least one chain, and this polymer comprises at least one polymeric sequence whose glass transition temperature (T_{g}) is between −150 and −20° C., preferably between −150 and −30° C. and more preferably between −150 and −40° C., the relative proportion by weight of this polymeric sequence being greater than 50%, preferably greater than 55% and even more preferably greater than 60% relative to the total weight of the polymer.

15. The composition as claimed in claim 14, characterized in that the cosmetically acceptable medium comprises one or more suitable solvents chosen from water, ketones, alcohols, alkylene glycols, alkylene glycol ethers, C₂₋₇ alkyl acetates, ethers, alkanes, aromatic hydrocarbons, aldehydes and volatile oils.

16. The cosmetic composition as claimed in either of claims 14 and 15, characterized in that said cosmetically acceptable medium furthermore includes a fatty phase composed of fatty substances that are liquid or solid at room temperature, of animal, plant, mineral or synthetic origin.

17. The cosmetic composition as claimed in any one of claims 14 to 16, characterized in that said cosmetically acceptable medium furthermore includes one or more anionic, cationic or nonionic, synthetic or naturally derived, film-forming polymers and/or one or more plasticizers.

18. The cosmetic composition as claimed in any one of claims 14 to 17, characterized in that said cosmetically acceptable medium furthermore includes a particulate phase consisting of pigments and/or nacres and/or fillers.

19. The cosmetic composition as claimed in any one of claims 14 to 18, characterized in that said physiologically acceptable medium furthermore includes one or more additives such as antioxidants, perfumes, essential oils, preserving agents, lipophilic or hydrophilic cosmetic active substances, hydrating agents, vitamins, dyes, essential fatty acids, sphingolipids, self-tanning agents, sunscreens, antifoam agents, sequestrants, antioxidants or free-radical scavengers.

20. The cosmetic composition as claimed in any one of claims 14 to 19, characterized in that it is in the form of an organic, aqueous or hydroalcoholic lotion, suspension, dispersion or solution, optionally thickened or gelled, a mousse, a spray, an oil-in-water, water-in-oil or multiple emulsion, a free, compact or cast powder, a solid or an anhydrous paste.

21. The cosmetic composition as claimed in any one of claims 14 to 20, characterized in that it is a hair lacquer or a hairstyling product.
